(19) United States
(12) Patent Application Publication
(10) Pub. No.: US 2013/0208957 A1
(43) Pub. Date: Aug. 15, 2013

(54) AUTOMATED QUANTIFICATION OF INTRAVASCULAR EMBOLIZATION SUCCESS

(71) Applicants: Steffen Gunther Wiesner, Koeln (NL); Matthias Bertram, Aachen (NL); Ming De Lin, Perry Hall, MD (US)

(72) Inventors: Steffen Gunther Wiesner, Koeln (NL); Matthias Bertram, Aachen (NL); Ming De Lin, Perry Hall, MD (US)

(73) Assignee: KONINKLIJKE PHILIPS ELECTRONICS N.V., EINDHOVEN (NL)

(21) Appl. No.: 13/700,496

(22) PCT Filed: May 19, 2011
(86) PCT No.: PCT/IB11/52194

§ 371 Date: Feb. 11, 2013

Related U.S. Application Data
(60) Provisional application No. 61/350,526, filed on Jun. 2, 2010.

(51) Int. Cl. A61B 5/00

(52) U.S. Cl. CPC A61B 5/0033
USPC 382/128

(57) ABSTRACT

The present invention relates to a device (2) for automatically quantifying intravascular embolization success, comprising a registration unit (4) adapted for registering a first image and a second image, a segmentation unit (6) adapted for segmenting a tissue of interest in the first image and in the second image and an evaluation unit (8) for evaluating a deviation of perfusion of the tissue of interest by comparing the first image and the second image. The first image is obtained before an interventional treatment, whereas the second image is obtained after such a treatment. Evaluating may comprise comparing the segments of the first and the second images and thus providing a quantitative measure for a perfusion deviation of the tissue, e.g. the perfusion deviation of a tumorous tissue before and after an embolization treatment.

### FIELD OF THE INVENTION

The invention relates to imaging systems which can be used for obtaining image data of tissues. In particular, the invention relates to a device and a method for automatically quantifying intravascular embolization success and to an imaging system capable of conducting such a method.

### BACKGROUND OF THE INVENTION

Transcatheter targeted chemoembolic drug delivery today represents the mainstay treatment of liver cancer, requiring intensive support by X-ray imaging with catheterization laboratory systems. Performing and analyzing a dual phase scan prior to an actual treatment, which dual phase scan comprises imaging during both the early-arterial and late-arterial phases of enhancement under utilization of an intra-arterially injected contrast agent for enhancement of vessels or tissue, e.g. tumorous tissue, the information from the dual phases allows visualization of e.g. a tumor-feeding vessel tree and a tumor itself. This may thereby facilitate identifying and reaching an optimal point of drug delivery and provides insight into the tumor dynamics.

While transcatheter arterial chemoembolization (also known as “TACE”) using lipiodol is currently the treatment of choice for palliative management of unresectable liver cancer, more recent techniques using drug-eluting beads or radioactive microspheres as the injected embolizing material are becoming increasingly popular, promising to yield higher treatment success while reducing side effects to the patient and thus shortening the required length of stay in the hospital. Such a drug-eluting bead based treatment may become more important than TACE as the prevalent treatment of the future.

However, in these methods of treatment, the technical success of the intervention cannot be verified as simple as in a traditional lipiodol TACE treatment, because neither the drug-eluting beads nor the radioactive microspheres are radio-opaque and so the drug delivery cannot readily be monitored in all situations as is done during and after a lipiodol-based TACE treatment. The necessity to track the positions where beads are delivered is especially important for measuring the treatment efficacy. Further, it is mandatory to consider that any bead reflux or washout from a tumor site may produce very severe side-effects.

### SUMMARY OF THE INVENTION

A judgement of success of treatment based on visual comparison of pre- and post-interventional CT or dual phase scans alone may only provide a subjective and qualitative measure. Furthermore, this approach of outcome control is complicated by the general difficulty in defining e.g. an exact outline of a tumor, by the occurring patient movement between pre- and post-treatment scans, and by the different appearance of the untreated and embolized tumors.

In particular, identifying the exact outline of a tumor is usually more difficult in the post-interventional scan due to its embolized state and the consequent under-perfusion. These issues make it difficult to relate and compare corresponding tumor regions within images before & after the treatment.

It would therefore be advantageous to achieve an automated and reliable quantification of intravascular embolization success, e.g. for use in imaging systems. It would be advantageous to provide a reliable and precise method for measuring the treatment efficacy of a transarterial chemoembolization treatment as described above.

To better address one or more of these concerns, in a first aspect of the invention there is provided a device for automatically quantifying intravascular embolization success.

The device comprises a registration unit adapted for registering a first image and a second image, wherein the first image is preferably obtained at a late-arterial phase before an intravascular embolization treatment and wherein the second image is preferably obtained at a late arterial phase after an intravascular embolization of a region of interest of a tissue to be examined. Registering the first and second images compensates shape deviations in the images induced by a patient and/or organ movement occurring between obtaining the two images. Preferably, the registering process is automatic.

The device further comprises a segmentation unit adapted for segmenting a tissue in the first image and in the second image. Thus, a direct comparison between a pre- and a post-treatment phase of a tissue can be accomplished. Due to the prior registration of these images, the segmentation only needs to be performed on one image. This may preferably be the pre-treatment image, which usually offers a better visibility of the tissue of interest. The segmentation may then also be used for the secondly acquired image as well. As proposed above, the registration is performed prior to the segmentation, which itself is only performed on one of the images. Depending on the visibility of the outline of the tissue of interest in the first image before treatment, the segmentation can alternatively be performed on both images independently. In this case, the segmentation result could in turn be used to support the registration process.

It is to be noted that the above features are not only directed to tumor tissues. In fact, intravascular embolization of a tumor tissue is a relevant application. Nevertheless, the above features also lead to an improved outcome control also for any other tissue structure of interest.

Further, the device comprises an evaluation unit for evaluating a perfusion of the tissue based on comparing the first image and the second image. The observed image enhancement within the tissue resulting from a contrast agent injected during the image acquisition, wherein the enhancement may be measured in Hounsfield Units (“HU”), may then quantitatively be assessed and compared between the pre- and post-interventional images and/or with the respective healthy tissue. This may result in a multitude of measures for an embolization success. Most of the conceivable embodiments of this invention differ mainly in the type of measure derived.

It is noted, that the first image and the second image are obtained under utilization of a contrast agent for enhancement of vessels and tissue of interest. Basically, the device according to the invention may be suitable for any image acquisition apparatus that is capable of obtaining an image of tissues of interest that are enhanced in comparison to healthy tissue due to the utilization of contrast agent uptake in a way that is different between pre- and post-intervention scans.

The gist of the invention lies in acquiring pre- and post-interventional images, e.g. by a CT or a dual phase scan under utilization of a contrast agent to enhancement of vessels and tissues and conducting the further steps of registering two respective late arterial phase images, segmenting the tissue and quantitatively evaluating and comparing the perfusion of the tissue before and after the treatment.

According to another aspect of the invention the evaluation unit is adapted for comparing voxels in the first image and voxels in the second image based on absolute or relative HU-enhancement values. The evaluation unit thereby determines the differences between the first image and the second image concerning the voxel-wise absolute or relative enhancement, thus providing information about perfusion deviations in the tissue due to the intervention.

According to another aspect of the invention, the evaluation conducted by the evaluation unit may be based on the number of voxels inside a tissue region which show HU-values above a given threshold and may therefore be considered as being perfused. The pre- and post-treatment difference in perfused tissue volumes is a direct measure of the embolization success. The given threshold value may be derived from a healthy tissue nearby the segmented tissue or alternatively, a fixed patient-independent threshold adjusted to the imaging setup and protocol may be used. A reference region may be defined automatically by the evaluation unit, the segmentation unit or the registration unit. This may also be conducted semi-automatically or manually.

According to yet another aspect of the invention the evaluation unit is adapted for determining a first HU-enhancement distribution in the first image, for determining a second HU-enhancement distribution in the second image and for determining the difference between the first HU-enhancement distribution and the second HU-enhancement distribution. The HU-enhancement distribution inside the tissue may be quantified e.g., using a histogram or the mean value and be compared between before and after the treatment. For better comparison, the data may be normalized by mean values derived from the respective reference regions in healthy tissue, obtained similar to the first embodiment. Thereby the evaluation unit provides information about a general perfusion change rate from a pre- to post-treatment time.

According to another aspect of the invention the evaluation unit is adapted for determining a direct voxel-to-voxel difference between the tissue regions before and after treatment. Since both images have been registered, the calculated difference may provide meaningful information about the spatial distribution of the change in perfusion within the tissue in response to the treatment.

Obtained information about perfusion deviations may be visualized in a color-coded fashion on a screen and may also comprise calculated volume fractions.

According to yet another aspect a method for automatically quantifying intravascular embolization success is provided. The method according to the invention basically comprises the steps of obtaining a first (pre-interventional) image of a region of interest of a tissue, obtaining a second (post-interventional) image of the region of interest of the tissue, registering the first image and second image, segmenting a tissue of interest in the first image and in the second image and evaluating the perfusion of the tissue based on comparing the first image and the second image.

For improving the results of the method according to the invention the first image and the second image are obtained in a late arterial phase each, hence increasing the visibility of the tissue of interest.

According to still another aspect of the invention, the segmented tissue regions of the first image and of the second image are compared with a healthy reference tissue.

According to a still further aspect of the invention the evaluation includes determining a first number of voxels in the first image comprising a HU-value above a given threshold, determining a second number of voxels in the second image comprising a HU-value above a given threshold and determining the difference between the first number and the second number of voxels.

According to another aspect of the invention the evaluation includes determining a first HU-enhancement distribution in the first image, determining a second HU-enhancement distribution in the second image and determining the difference between the first HU-enhancement distribution and the second HU-enhancement distribution.

Further, according to another aspect of the invention the evaluation includes determining a direct voxel-to-voxel difference between the first image and the second image.

In another exemplary embodiment of the present invention a computer program or a computer program element is provided, which computer program element is a part of a computer program adapted for controlling a device, e.g. an imaging apparatus, according to one of the above described aspects, which, when being executed by a processing unit, is adapted to perform corresponding method steps according to the invention.

The computer program element may therefore be stored on a computing unit, a calculating device, an electronic device, which may also be part of an embodiment of the present invention. The computing unit may be adapted to perform or initiate a performing of method steps associated with the above described device. Moreover, it may be adapted to operate the components of the above described device. A computer program element may be loaded into a working memory of a data processor, which data processor may thus be equipped to carry out the method according to the invention.

This exemplary embodiment of the invention covers both, a computer program element that is adapted for using the invention right from the start and a computer program element that is adapted for using the invention through being integrated by means of an update to turn an existing program into a program that uses the invention.

Further, the computer program element may also be able to provide all necessary steps to fulfill the procedure of an exemplary embodiment of the method according to the invention as described above.

According to a further exemplary embodiment of the present invention, a computer readable medium, such as a CD-ROM or the like, is presented wherein the computer readable medium has a computer program element stored on it, which computer program element is described by the preceding section.

However, the computer program element may also be presented over a network like the World Wide Web and may be downloadable into the working memory of a data processor from such a network.

According to a further exemplary embodiment of the present invention, a medium for making a computer program element available for downloading is provided, which computer program element is adapted to perform a method according to one of the previously described embodiments of the invention.

Additional embodiments deriving from additional quantitative measures of tissue perfusion/embolization extracted from the registered and segmented images are conceivable and are understood to be included in the invention described in this disclosure.

Furthermore, it is to be noted that the outcome control of the treatment could be performed offline and outside of the intervention room.

The described functionality for quantitative outcome control is potentially relevant not only for all kinds of targeted drug delivery but also for other radiological interventions (e.g. uterine fibroid or uterine artery embolization, embolization of GI bleedings) that are or may be performed in a catheter laboratory using dual phase scans, CT imaging or any modality that utilizes a contrast agent for enhancement of a tissue in such a way that the enhancement is different before and after the treatment.

It has to be noted that exemplary embodiments of the invention are described with reference to different subject matters. In particular, some exemplary embodiments are described with reference to apparatus type claims whereas other exemplary embodiments are described with reference to method type claims. However, a person skilled in the art will gather from the above and the following description that, unless otherwise notified, in addition to any combination of features belonging to one type of subject matter also any combination between features relating to different subject matters, in particular between features of the apparatus type claims and features of the method type claims, is considered to be disclosed with this application. However, all features can be combined providing synergetic effects that are more than the simple summation of the features.

### BRIEF DESCRIPTION OF THE DRAWINGS

The aspects defined above and further aspects, features and advantages of the present invention can also be derived from the examples of embodiments to be described herein after and are explained with reference to examples of embodiments, but to which the invention is not limited. The invention will be described in more detail hereinafter with reference to the drawings.

FIG. 1 shows a schematic overview of a device according to the invention.

FIGS. 2a and 2b show late arterial phases of dual phase scans taken before (2a) and after (2b) treatment with drug-eluting beads.

FIGS. 3a and 3b show extracted tumor regions by means of segmentation applied to the pre-interventional image.

FIGS. 4a and 4b show pre- and post-intervention enhanced tumor regions normalized by taking the difference to the mean value of the corresponding reference region.

FIG. 5 shows a method according to the present invention in a block diagram.

FIG. 6 is a block diagram of an imaging system according to the present invention.

### DETAILED DESCRIPTION OF EMBODIMENTS

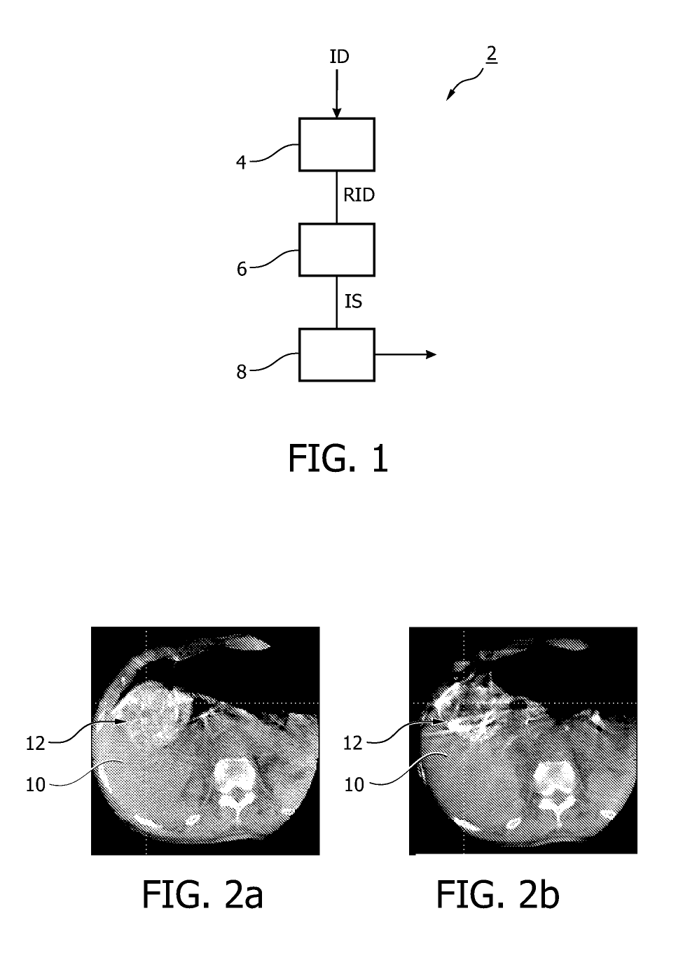

A first exemplary embodiment will be described in the following. FIG. 1 schematically shows a device 2 for automatically quantifying intravascular embolization success according to the first embodiment of the invention. The device 2 comprises a registration unit 4 which is provided with image data ID. This image data comprises a first image obtained before an intravascular embolization treatment is conducted and a second image obtained after an intravascular embolization treatment is conducted. The registration unit 4 registers the first image and the second image in order to compensate for patient/organ motion occurring between the acquisition time of the first image and the second image and provides registered image data RID. Registering itself may include selecting one or more reference points of the first image and of the second image and aligning these reference points relative to each other, thereby turning and stretching at least one of the first image and the second image. The original image data ID may be provided by an imaging system not shown in FIG. 1.

The device 2 further comprises a segmentation unit 6 which is adapted for segmenting a tissue of interest in the first image and in the second image provided by the registration unit 4, e.g. using well known segmentation methods. It may be beneficial to conduct a first segmentation process with the first image only and use the first segmentation process also for the second image, since the first image and the second image are fully aligned by the registration process and the first image may provide a better visibility of the tissue of interest, e.g. a tumorous tissue.

The device 2 further comprises an evaluation unit 8, which is provided with segments IS of the first image and the second image by the segmentation unit 6 containing tissue image parts of the first image and of the second image, e.g. tumorous image parts. The evaluation unit 8 is adapted for comparing these segments and thereby allowing to evaluate a perfusion of the tissue of interest. Comparing may include a voxel-by-voxel comparison of the segments and conduct a color-coding for differences between the segments, depending on the size of the differences. Additionally or as an alternative, comparing may also include comparing a HU-enhancement distribution of the segments. For a better comparison result, normalization of the image segments data may be accomplished. Further, additionally or as an alternative, comparing may include counting the number of voxels in each of the image segments data showing HU-values above a given threshold and then compared. The voxels that reach above a given threshold may be considered as perfused. Perfused areas may be shown in different colors on a screen attached to the device 2 according to the invention.

As an example for a tissue of interest in FIG. 2a a tissue 10 with a tumorous region 12 is depicted before a treatment with drug-eluting beads is conducted. Therefore, the image shown in FIG. 2a will be referred to as “first image”. The first image is obtained at a late arterial phase, which may begin a short duration after a contrast medium has been transmitted and provides a best contrast between the tumorous region 12 and normal tissue.

In FIG. 2b the tissue 10 is depicted after the treatment with drug-eluting beads has been conducted and is therefore referred to as “second image”. The first image and second image are registered and it is clearly visible that the enhancement within the tumor region 12 in FIGS. 2a and 2b differ.

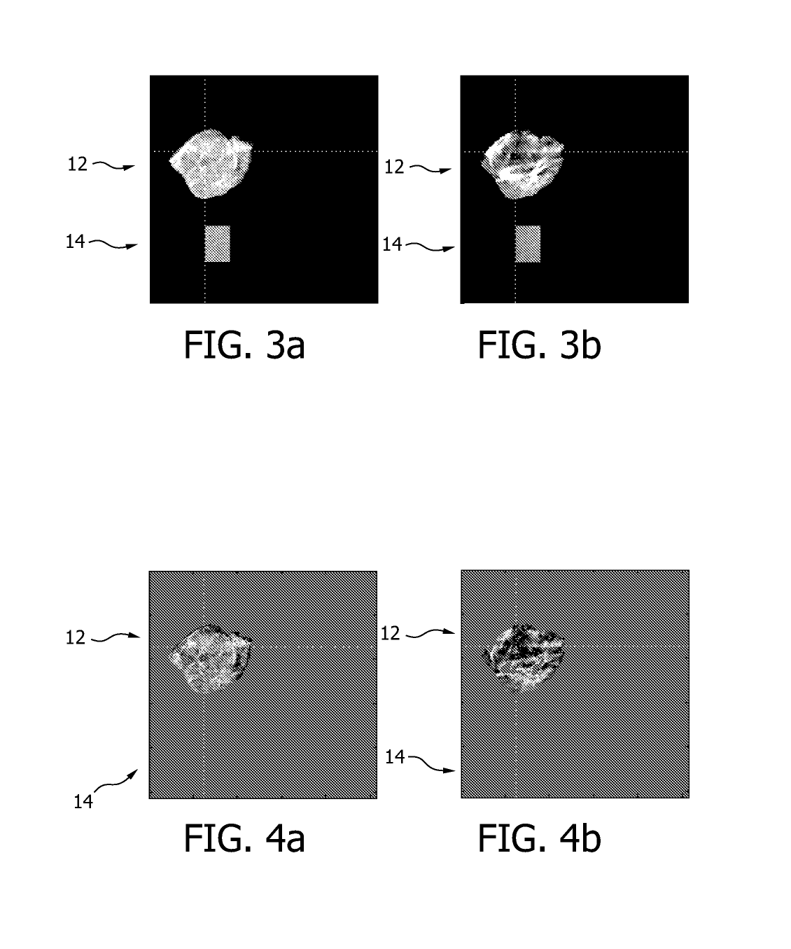

For a better ability to judge the treatment success of the intravascular embolization a segmentation of the tumor region 12 is conducted, as can be seen in FIGS. 3a and 3b. Such a segmentation is directed to partitioning image data into contiguous regions representing individual anatomical objects and is usually a prerequisite for further investigations. Segmentation may be conducted manually in a rather time-consuming process, or by an automatic process. In either way the segmentation process depends on the certain kind of examined tissue and may comprise different methods already known to a person skilled in the art. Therefore, the segmentation process is not described in detail.

A measure to judge the treatment success can be obtained by comparing the tumor regions 12 from the FIGS. 3a and 3b since the tumor region in FIG. 3b is less perfused than the tumor region 12 of FIG. 3a. The comparison may be achieved by different method steps already mentioned in the description part for FIG. 1. If the comparison method includes comparing HU-values of the tumor region 12 with a predetermined HU-value or HU-interval, a reference section 14 that is outside the tumor region 12 may be defined in the first image and/or the second image, as indicated in FIGS. 3a and 3b. These reference regions comprise a mean HU-value.

For directly obtaining information about tissue regions being hypo- or hyper-perfused the image sections containing the tumor region 12 may be normalized, e.g. conducting a histogram normalization by taking the difference to the mean value of the corresponding reference region. In FIGS. 4a and 4b the normalized pre- and post-interventional segmented images are shown and it is clearly visible that the background now has the same shading as the reference area 14, hence rendering the reference area invisible. Regions with a brighter shading can now be considered hyper-perfused, wherein regions with a darker shading represent hypo-perfusion.

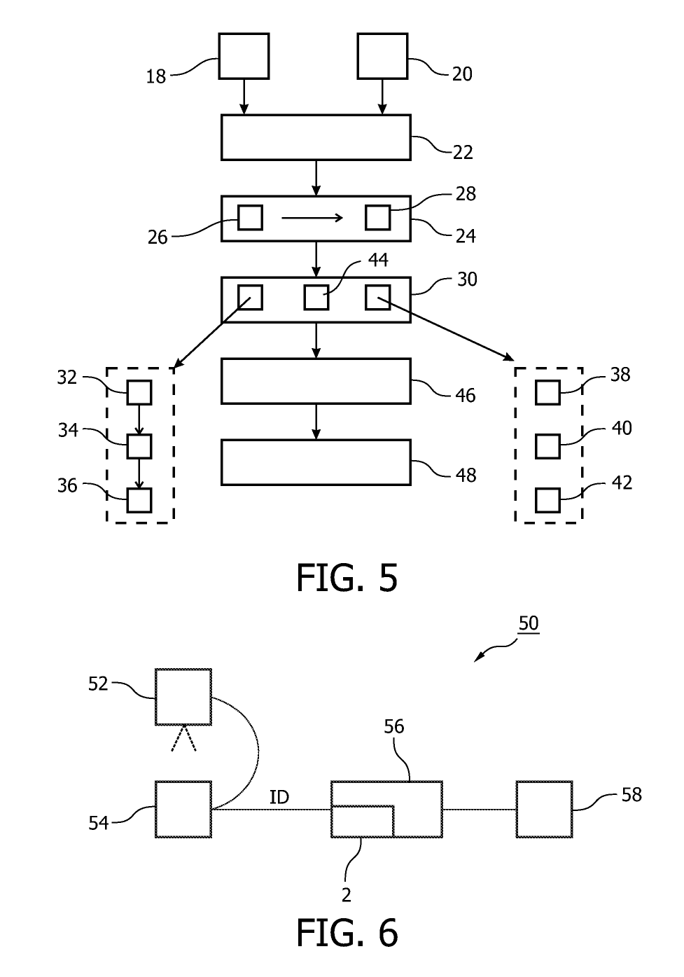

FIG. 5 shows a method for automatically quantifying intravascular embolization success according to the invention in a schematic block diagram. First of all, a first image of a region of interest of a tissue 10 is obtained 18. Later on, after the intravascular embolization has been conducted, a second image of the region of interest of the tissue is obtained 20. For enabling a comparison, the first image and second image are the registered 22. Afterwards, a tissue of interest in the first image and in the second image is segmented 24. The segmentation process, as it may be cumbersome, may conducted for the first image only 26, since there the tissue of interest may comprise a better visibility. Then, the already determined segmentation borders may then be applied to the second image 28. Afterwards, the perfusion of the tissue of interest may then be evaluated 30 based on comparing the first image and the second image.

The evaluation may comprise determining 32 a first number of voxels in the first image comprising a HU-value above a given threshold, determining 34 a second number of voxels in the second image comprising a HU-value above a given threshold and determining 36 the difference between the first number and the second number of voxels.

The evaluation may additionally or as an alternative comprise determining 38 a first HU-enhancement distribution in the first image, determining 40 a second HU-enhancement distribution in the second image and determining 42 the difference between the first HU-enhancement distribution and the second HU-enhancement distribution.

Further, the evaluation may also comprise determining 44 a direct voxel-to-voxel difference between the first image and the second image.

For improving the visibility of the evaluation, the differences in perfusion may be highlighted by visualizing 46 the difference through applying a suitable and unambiguous color-code.

Finally, the evaluated difference in perfusion may be output 48 to a screen or any other suitable output device.

An exemplary embodiment of an imaging system will be described in the following. FIG. 6 is a block diagram of an imaging system 50 according to the invention. The imaging system 50 comprises a radiation source 52, a radiation detection module 54, a data processing unit 56 with a central processing unit, a memory and a device 2 according to the first embodiment of the invention, described in regard to FIG. 1.

Preferably the imaging system 50 further comprises a display unit 58, which display unit is connected with the data processing unit 56. The device 2 receives image data ID from the radiation detection module 54.

Merely as an example, but not limiting thereto, in FIG. 6 the radiation source 52 and the radiation detection module 54 are part of a C-arm system and the radiation source 52 therefore is an X-ray source and the radiation detection module is an X-ray detection module. The radiation source 52 and the radiation detection module 54 may also be part of a computer tomography system rotating around an object or subject to be examined.

In the claims, the word “comprising” does not exclude other elements or steps, and the indefinite article “a” or “an” does not exclude a plurality. A single processor or other unit may fulfill the functions of several items re-cited in the claims. The mere fact that certain measures are re-cited in mutually different dependent claims does not indicate that a combination of these measures cannot be used to advantage.

A computer program may be stored and/or distributed on a suitable medium, such as an optical storage medium or a solid state medium supplied together with or as part of other hardware, but may also be distributed in other forms, such as via the internet or other wired or wireless telecommunication systems.

It has to be noted that embodiments of the invention are described with reference to different subject matters. In particular, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

Any reference signs in the claims should not be construed as limiting the scope.

1. A device (2) for automatically quantifying intravascular embolization success, comprising
a registration unit (4) adapted for registering a first image and a second image;
a segmentation unit (6) adapted for segmenting a tissue in the first image and in the second image; and
an evaluation unit (8) for evaluating a deviation of perfusion of the tissue by comparing the first image and the second image,
wherein the first image supplied to the device (2) is obtained before an intravascular embolization treatment and wherein the second image supplied to the device (2) is obtained after an intravascular embolization of a region of interest of a tissue to be examined.

2. The device (2) according to claim 1,
wherein the evaluation unit (8) is adapted for comparing voxels of the first image with voxels of the second image based on absolute or relative HU-enhancement values.

3. The device (2) according to claim 1,
wherein the evaluation unit (8) is adapted for determining a first HU-enhancement distribution in the first image, determining a second HU-enhancement distribution in the second image and determining the difference between the first HU-enhancement distribution and the second HU-enhancement distribution.

4. The device (2) according to claim 1,
wherein the tissue is a tumor tissue (12).

5. An imaging system (50), comprising:
a radiation source (52);
a radiation detection module (54);
a data processing unit (56) comprising a central processing unit, a memory, and a device (2) according to claim 1; and
a display unit (58) connected with the data processing unit (56);
wherein the device (2) receives image data (ID) from the radiation detection module (54).

6. A method for automatically quantifying intravascular embolization success, comprising the steps:
obtaining (18) a first image of a region of interest of a tissue (12);
obtaining (20) a second image of the region of interest of the tissue (12);
registering (22) the first image and second image;
segmenting (24) the tissue of interest in the first image and in the second image; and
evaluating (30) the perfusion of the tissue based on comparing the first image and the second image,
wherein the first image is obtained before an intravascular embolization treatment and wherein the second image is obtained after an intravascular embolization.

7. The method according to claim 6,
wherein the first image and the second image are obtained in a late-arterial phase each.

8. The method according to claim 6,
wherein segmenting (24) is only performed (26) on the first image and is further used (28) for the second image.

9. The method according to claim 6, wherein evaluating (30) includes
comparing the segmented region of the first image and of the second image with a healthy reference tissue.

10. The method according to claim 6, wherein evaluating includes
determining (32) a first number of voxels in the first image comprising a HU-value above a given threshold;
determining (34) a second number of voxels in the second image comprising a HU-value above a given threshold;
determining (36) the difference between the first number and the second number of voxels.

11. The method according to claim 6, wherein evaluating includes
determining (38) a first HU-enhancement distribution in the first image;
determining (40) a second HU-enhancement distribution in the second image;
determining (42) the difference between the first HU-enhancement distribution and the second HU-enhancement distribution.

12. The method according to claim 6, wherein evaluating (30) includes
determining a direct voxel-to-voxel difference between the first image and the second image.

13. The method according to claim 9, further comprising the step of visualizing (46) the difference.

14. The method according to claim 6, wherein obtaining the image includes conducting a contrast enhanced scan.

15. A computer program element for controlling a device (2) according to claim 1.

16. A computer readable medium having stored the program element of claim 15.
